(19) United States
(12) Patent Application Publication
(10) Pub. No.: US 2012/0289810 A1
(43) Pub. Date: Nov. 15, 2012

(54) PRESSURE MONITOR

(76) Inventor: Timothy J. Ehrecke, Davenport, IA (US)

(21) Appl. No.: 13/549,862

(22) Filed: Jul. 16, 2012

Related U.S. Application Data
(63) Continuation-in-part of application No. 12/368,072, filed on Feb. 9, 2009.
Application No. 12/368,072 is a continuation-in-part of application No. 12/090,068, filed on Dec. 30, 2008, now abandoned.

(51) Int. Cl. A61B 3/16

(52) U.S. Cl. 600/398

(57) ABSTRACT

A bottom surface with an adhesion surface including a plurality of micro-protrusions, or an adhesive adhered to the bottom surface; a pressure monitoring device configured to measure intraocular pressure of an eye and generate a signal representing the measured pressure; and a transmitter configured to transmit the signal. The bottom surface is arranged to secure the sensor to: an outer surface of a conjunctiva for the eye without penetrating the conjunctiva; or an outer surface of a sclera for the eye without penetrating the sclera.

### CROSS REFERENCE TO RELATED APPLICATIONS

This is a continuation-in-part patent application under 35 USC 120 of U.S. patent application Ser. No. 12/368,072, filed Feb. 9, 2009, which is a continuation-in-part application under 35 USC 120 of U.S. patent application Ser. No. 12/090,068, filed Dec. 30, 2008, which application claims the benefit under 35 U.S.C. §119(e) of U.S. Provisional Application 61/063,923 filed on Feb. 7, 2008, which applications are incorporated herein by reference.

### TECHNICAL FIELD

The present invention relates to a system and method for measuring physiological parameters in organisms, and is particularly directed to a system and method for measuring intraocular pressure in the eye.

### BACKGROUND

Glaucoma patients and post-operative patients of eye surgery require regular monitoring of the intraocular pressure (IOP) of their eyes in order to diagnose degenerative conditions that may lead to degraded sight and/or blindness without immediate medical treatment. Accordingly, such patients must make frequent trips to their ophthalmologist's office for this regular monitoring of their IOP with conventional mechanical impact type tonometers. This becomes a nuisance to the patient after a time, leading to patient resistance to compliance. In addition, the only measurement of the patient's IOP that the doctor may use for diagnosis is the pressure that exists at the time of the office visit. Therefore, if the pressure is normal at the time of the visit, but becomes high thereafter, the patient's actual risk of blindness may be misdiagnosed. Also, if the pressure measured at the time of the office visit is high for reasons other than eye degeneration, the patient may be falsely diagnosed and be required to undergo therapy or take medication that may not be needed.

Intraocular pressure has been known to fluctuate widely during, any given period of time and thus, should be monitored many times during the period of a day in order to gain an average or representative IOP, which in turn may be tracked for diagnosis. Attempts have been made to permit glaucoma patients to monitor their IOP at home many times during the period of a day with a self-tonometry portable instrument. Reference is made to the paper “Self-Tonometry to Manage Patients with Glaucoma and Apparently Controlled Intraocular Pressure”, Jacob T. Wilensky et al., published in Arch Opthalmol, Vol. 105, August 1987 for more details of such a device. This paper describes a portable tonometer instrument consisting of a pneumatically driven plunger fitted with an elastic membrane that slowly comes forward and applanates the cornea. Applanation is detected by an internal optic sensor and the pressure necessary to achieve applanation is registered and displayed automatically. The patient is able to prepare the eye and self-tonometer and activate the instrument for taking the measurement. However, the device proposed is relatively large and bulky and not conducive to convenient transport with the patient during normal daily routine in order to measure IOP. In addition, the proposed technique requires special eye preparation by instilling a topical anesthetic in the eye prior to tonometric measurements.

Also, very crude attempts have been made to develop methods of non-invasively monitoring IOP using passive electronic circuitry and radiotelemetry disposed at the eye. In the papers of R. L. Cooper et al. namely, those published in Invest, Opthalmol Visual Sci., pp. 168-171, February 1977; British JOO, 1979, 63, pp. 799-804; Invest, Opthalmol Visual Sci., 18, pp. 930-938, September, 1979; and Australian Journal of Opthalmology 1983, 11, pp. 143-148, a miniature guard ring applanating transsensor (AT) that included electronic components that changed in resonance proportional to the IOP was mounted in an acrylic or sauflon haptic contact lens element that was individually designed for the human eye. The AT was mounted in the lower part of the scleral haptic so that it applanated the inferior sclera under the lower lid. The whole haptic ring was placed in the conjunctival formix. Intraocular pressure was monitored from the AT with an automatic continual frequency monitor (ACFM) attached by adhesive and elastic bands to the exterior of the lower eye lid. The ACFM induced in the AT electromagnetic oscillations at varying radio frequencies via a magnetic coupling of inductive coils and monitored for its resonant frequency, which is representative of IOP. This device is clearly uncomfortable and bulky, minimizing expected patient compliance. In addition, the device measures IOP by applanation of the sclera, which is a rather unconventional method of measuring IOP.

In another paper reported in Investigative Opthalmology Reports, pp. 299-302, April, 1974 by B. G. Gilman, a device is presented for measuring the LOP of a rabbit in a continuous manner with strain gauges mounted (embedded) in soft, flush fitting, silastic gel (hydrogel) contact lenses. The exact shape of the eye of the rabbit was obtained by a molding procedure. Leads of the strain gauges extended from the lens and were connected to a wheatstone bridge arrangement for measurement taking. The paper suggests that the embedded strain gauges may be used with a miniature telemetry package completely contained in a hydrophilic hydrogel contact lens for continuous, noninvasive, long duration monitoring of IOP, although no design was provided. This device proposes wire connections for telemetry, which requires that wires be placed adjacent the eye under the eyelid. Also, the proposed approach requires the molding of a special contact for each individual eye, a practice which would make widespread use unattractive and expensive.

In 1993, an IEEE paper was presented by C. den Besten and P. Bergveld of the University of Twente, The Netherlands, proposing a new instrument for measuring area of applanation entitled “A New Tonometer Based on Application of Micro-Mechanical Sensors.” This new instrument is based on the Mackay-Marg principle of tonometer operation in which a plate having a diameter of six millimeters or less is pressed against and flattens a portion of the cornea of the eye, referred to as “applanation.” In the middle of the plate is a small pressure sensitive area that is pressed against the flattened portion of the cornea with a slowly increasing force while the pressure area is electronically measured. The applanation sensor of this new instrument comprises a micro-machined plunger and pressure sensing electronics on three electrically insulated levels of a silicon substrate resulting in a modified Mackay-Marg tonometer in which the radius of the flattened area and the distance between the periphery of the applanation and the pressure center can be measured to render a more accurate pressure area measurement. In the work presented in this paper, the researchers did not actually propose a pressure sensor or transducer. In addition, it is not clear if, for as long as the eye is applanated, there is a need to know the area of applanation. Sufficient applanation is usually determined by the difference in trough height from the peak to dip of the pressure profile. The dip is unlikely to occur unless sufficient applanation is achieved.

Also, in U.S. Pat. No. 5,830,139 entitled “Tonometer System for Measuring Intraocular Pressure by Applanation and/or Indentations,” issued to Abreu on Nov. 3, 1998, a tonometer system is disclosed. The system uses a contact device shaped to match the outer surface of the cornea and having a hole through which a movable central piece is slidably disposed for flattening or indenting a portion of the cornea. A magnetic field controls the movement of the central piece against the eye surface to achieve a predetermined amount of applanation. A sophisticated optical arrangement is used to detect when the predetermined amount of applanation has been achieved to measure IOP, and a calculation unit determines the IOP based on the amount of force the contact device must apply against the cornea in order to achieve the predetermined amount of applanation. The magnetic and optical arrangement of this device requires special alignment and calibration techniques rendering it difficult for use as a self-tonometry device. See also U.S. Pat. No. 7,169,106 issued to Fleishman et al. entitled “Intraocular Pressure Measurement Including a Sensor Mounted in a Contact Lens.”

Other systems have been developed to detect multiple parameters using contact devices placed against the surface of the eye. For example U.S. Pat. Nos. 6,423,001 and 7,041,063, both issued to Abreu and incorporated by reference herein in their entireties, disclose various apparatuses for measuring IOP using contact devices placed on the surface of the eye. However, the devices disclosed by Abreu all float on the surface of the eye, and are therefore prone to displacement or movement in the same manner as a conventional contact lens for vision correction.

Because IOP varies over the course of the day based on a number of factors, it is advantageous to accurately measure IOP over several hours to compile a range. of readings to account for normal variations. As discussed further in the article “Diurnal Variations in Intraocular Pressure” by J. T. Wilensky.sup.1, which is incorporated by reference herein, some of these factors act over periods ranging from seconds to minutes or hours; others act over a longer duration. .sup.1 Trans Am Opthalmol Soc. 1991; 89: 757-790.

While the various foregoing described U.S. patents and papers propose various devices and instruments for tonometry, none appears to offer a viable, inexpensive, and convenient solution to the immediate problem of self-tonometry. The present disclosure overcomes the drawbacks of the proposed instruments described above to yield a simple, inexpensive, and easy-to-use instrument that completely automates the tonometry process and offers post-processing of tonometer IOP readings from which a proper evaluation and diagnosis by an ophthalmologist may be performed.

The prior art, including the cited patents and publications, all of which are incorporated by reference herein, has failed to teach an IOP device that is comfortable to wear; allows for continuous monitoring; does not restrict vision, and may be inserted in the eye cavity for extended periods of time to assist with capturing and collecting diurnal variations in IOP as well as systemic variations in IOP.

### SUMMARY

According to aspects illustrated herein, there is provided a sensor including: a bottom surface with an adhesion surface including a plurality of micro-protrusions, or an adhesive adhered to the bottom surface; a pressure monitoring device configured to measure intraocular pressure of an eye and generate a signal representing the measured pressure; and a transmitter configured to transmit the signal. The bottom surface is arranged to secure the sensor to: an outer surface of a conjunctiva for the eye without penetrating the conjunctiva; or an outer surface of a sclera for the eye without penetrating the sclera.

According to aspects illustrated herein, there is provided a sensor including: an adhesion surface with a plurality of micro-protrusions; a pressure monitoring device configured to measure intraocular pressure of an eye and generate a signal representing the measured pressure; and a transmitter configured to transmit the signal. The adhesion surface is arranged to secure the sensor to an outer surface of a conjunctiva for the eye without penetrating the conjunctiva.

According to aspects illustrated herein, there is provided a method for measuring intraocular pressure using a sensor including a bottom surface with an adhesion surface with a plurality of micro-protrusions or an adhesive adhered to the bottom surface, a pressure monitoring device, and a transmitter, the method including: securing, with the bottom surface, the sensor to: an outer surface of a conjunctiva for an eye without penetrating the conjunctiva; or an outer surface of a sclera for an eye without penetrating the sclera; measuring, using the pressure monitoring device, intraocular pressure in the eye; generating a signal representing the measured pressure; and transmitting, using the transmitter, the signal.

### BRIEF DESCRIPTION OF THE DRAWINGS

Various embodiments are disclosed, by way of example only, with reference to the accompanying schematic drawings in which corresponding reference symbols indicate corresponding parts, in which:

FIG. 1 is a front view of an exemplary embodiment of the sensor adhered to the sclera of a human eye;

FIG. 2 is a perspective view of an exemplary embodiment of the sensor;

FIG. 3 is a top view of an exemplary embodiment of the sensor;

FIG. 4 is a simplified schematic diagram of some of the components of an exemplary embodiment of the sensor;

FIG. 5 is a simplified schematic diagram of some of the components of another exemplary embodiment of the sensor;

FIG. 6 is a simplified schematic diagram of some of the components of a third exemplary embodiment of the sensor;

FIG. 7 is a simplified schematic diagram of some of the components of an exemplary embodiment of a base unit for communication with the sensor;

FIG. 8 is a side view of a portion of an embodiment of a pressuring monitoring device having a variable capacitance and a variable inductance resonant circuit;

FIG. 9 is a perspective top view of a pressure monitor;

FIG. 10 is a perspective bottom view of the pressure monitor shown in FIG. 9;

FIG. 11 is a schematic representation of micro-protrusions;

FIG. 12 is a perspective view of the pressure monitor shown in FIG. 9 adhered to a human eye;

FIG. 13 is a cross-sectional view generally along line 13-12/13-12 in FIG. 12; and,

FIG. 13 is a cross-sectional view generally along line 13-12/13-12 in FIG. 12.

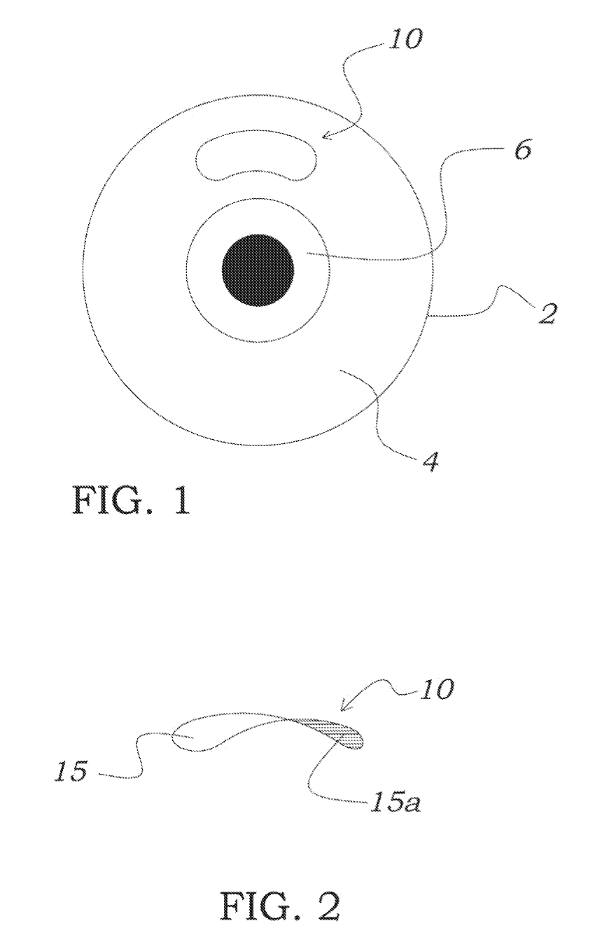

### DETAILED DESCRIPTION

At the outset, it should be appreciated that like drawing numbers on different drawing views identify identical, or functionally similar, structural elements of the disclosure. It is to be understood that the disclosure as claimed is not limited to the disclosed aspects.

Furthermore, it is understood that this disclosure is not limited to the particular methodology, materials and modifications described and as such may, of course, vary. It is also understood that the terminology used herein is for the purpose of describing particular aspects only, and is not intended to limit the scope of the present disclosure.

Unless defined otherwise, all technical and scientific terms used herein have the same meaning as commonly understood to one of ordinary skill in the art to which this disclosure belongs. It should be understood that any methods, devices or materials similar or equivalent to those described herein can be used in the practice or testing of the disclosure.

Before the various embodiments of the present invention are explained in detail, it is to be understood that the invention is not limited in its application to the details of construction and the arrangements of components set forth in the following description or illustrated in the drawings. The invention is capable of other embodiments and of being practiced or of being carried out in various ways. Also, it is to be understood that phraseology and terminology used herein with reference to device or element orientation (such as, for example, terms like “front”, back “up”, “down”, “top”, “bottom”, and the like) are only used to simplify description of the present invention, to and do not alone indicate or imply that the device or element referred to must have a particular orientation. In addition, terms such as “first”, “second”, and “third” are used herein and in the appended claims for purposes of description and are not intended to indicate or imply relative importance or significance.

Referring now to the drawings, wherein like reference numerals designate identical or corresponding parts throughout the several views, FIG. I illustrates a first exemplary embodiment of a sensor 10 adhered to the sclera 4 of a human eye 2. As shown in the various figures herein, the sensor 10 is shown with a smoothed hour-glass shape. However, the particular shape of the sensor 10 is in no way limiting, and therefore the sensor 10 may have any shape that is suitable for the particular application for that specific sensor 10. As shown in FIG. I , the sensor 10 may be adhered to the sclera 4 in a position on the eye 2 above the cornea 6. The sensor 10 may be adhered to other parts of the sclera 4 as well, although only a position above the cornea 6 is shown herein.

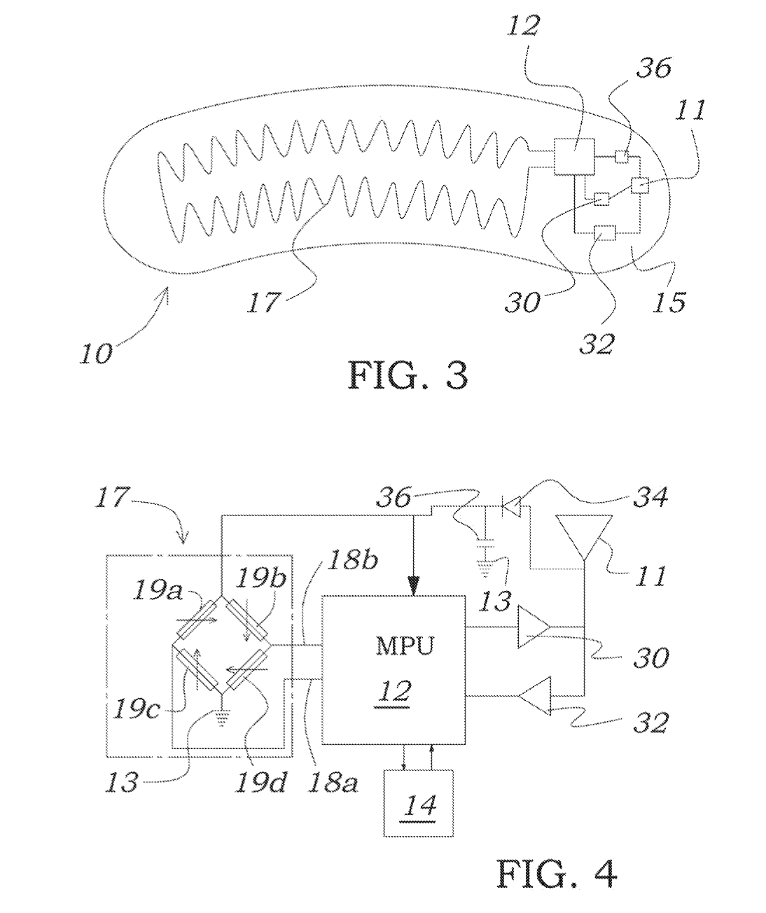

The sensor 10 includes a body 15, which is the medium to which all other components of the sensor 10 are affixed or embedded, as shown in FIGS. 2 and 3. At least a portion of the body 15 that abuts the sclera 4 includes an adhesion substrate 15a. The adhesion substrate 15a may take several embodiments depending on the application for the sensor 10 and/or materials used. For example, the adhesion substrate 15a may be a patch of biologically safe adhesive integrated into a portion of the body 15.

Alternatively the adhesion substrate 15 a may be manufactured separately from the sensor 10 and/or body 15, in which case the adhesion substrate 15a may be placed on the sclera 4 prior to the placement of the sensor 10. In this embodiment, the adhesion substrate 15a would be applied to the portion of the sclera 4 where the sensor 10 is desired. After the adhesion substrate 15a is applied, the sensor 10 may then be positioned on the sclera 4 over the adhesion substrate 15a. In this embodiment, the adhesion substrate 15a must be a material that is both adhesive to the surface of the eye 2 and to the portion of the body 15 of the sensor 10 adjacent the eye 2. The adhesion substrate 15a and material used to construct the body 15 are ideally biologically inert and should not be chemically active on the surface of the eye 2. Furthermore, the adhesion substrate 15a should be reversibly adhesive. Certain biologically safe adhesives that may be used as an adhesive substrate 15a and/or for use in construction of the body 15 include but are not limited to silicones, hydrogels, polymer hydrogels, or poly-methyl methacrylates.

A simplified schematic diagram for an exemplary embodiment of a smart sensor 10 is shown in FIG. 4. In that embodiment and sensor MPU 12 is embedded in the body 15 of the sensor 10. The sensor MPU 12 controls the operation of the sensor 10 and the various components thereof. The sensor MPU 12 may be any type of micro controller/processor, such as a programmable logic controller or other circuitry known to those skilled in the art that is capable of directing, Is communicating with, or controlling electromechanical devices. The embodiment shown in FIG. 4 does not include a battery 16 to power the sensor 10. Instead, a sensor antenna II configured as an inductive coil is in electronic communication with a diode 34 and power capacitor 36 that is sized to provide enough power to the sensor 10 for one interrogation cycle. The power capacitor 36 is in electronic communication with the sensor MPU 12 and is the power source therefor.

Also affixed and/or embedded within the body 15 is a pressure monitoring device. In the embodiment shown in FIG. 4, the pressure monitoring device is comprised of a strain array 17. The strain array 17 is positioned on the body 15 such that when the sensor 10 is adhered to the eye 2, the strain array 17 abuts a portion of the sclera 4. The strain array 17 shown in FIG. 4 is comprised of a first, second, third, and fourth resistor 19a, 19b, 19c, 19d that have variable resistance values. The first and third resistors 19a, 19c are connected in series, as are the second and fourth resistors 19b, 19d. The pair of first and third resistors 19a, 19c are connected in parallel with the pair of second and fourth resistors 19b, 19d. The electrical lead between the first and third resistors 19a, 19c is electronically connected to the sensor MPU 12 as is the electrical lead between the second and fourth resistors 19b, 19d. The strain array 17 also includes a ground 13. As the shape of the portion of the sclera 4 to which the sensor 10 is adhered changes, the resistance of the strain array 17 changes. The sensor MPU 12 may be programmed to detect the difference in resistance of the pair of first and third resistors 19a, 19c as compared to the resistance of the second and fourth resistors 19b, 19d, which is indicated strain array first output 18a and strain array second output 18b, respectively. This value may be stored in a flash memory 14 electronically connected to the sensor MPU 12, which flash memory 14 is affixed to and/or embedded in the body 15, or it may be transmitted to a base unit 40 as described in detail below. In other embodiments not pictured herein, a media for storing data other than flash memory 14 is used. Accordingly, the type of media used for storing the data in no way limits the scope of the present disclosure.

Besides acting as an inductive coil that charges the power capacitor 36, the sensor to antenna 11 may also enable transmitting/receiving data to/from the sensor 10. In the embodiment shown in FIG. 4, a transmitter 30 and a receiver 32 are affixed to and/or embedded in the body 15 and electronically connected to the sensor MPU 12. Accordingly, the sensor antenna 11 in combination with the receiver 32 may facilitate programming of the sensor MPU 12 through remote communication, such as electromagnetic energy. Furthermore, the sensor antenna 11 in combination with the transmitter 30 may facilitate downloading the data in the flash memory 14 if the sensor 10 is so equipped, or transmitting the instantaneous value recorded by the strain array 17 via electromagnetic energy, both of which are described in more detail below.

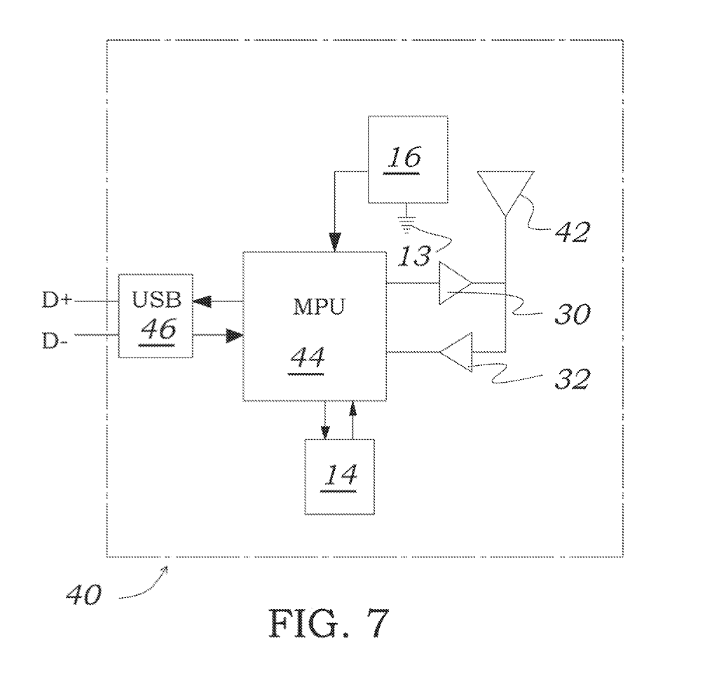

It is contemplated that the embodiment shown in FIG. 4 will be used as a smart passive sensor 10. Accordingly, a base unit 40 will be required to communicate with the sensor 10 to measure and record IOP. A simplified schematic diagram of one embodiment of a base unit 40 is shown in FIG. 7. As shown in FIG. 7, the base unit 40 includes a battery 16, flash memory 14, base MPU 44, transmitter 30, receiver 32, and base antenna 42, all of which are ultimately controlled by the base MPU 44. The base unit 40 may also include a USB interface 46 for communication with a computer (not shown). Other types of communication devices and/or methods may be used to enable data transmission between the base unit 40 and a computer (not shown). For example, wireless communications using infrared waves or radio waves may be used, as may other wired methods such as serial ports. Accordingly, the present disclosure is in no way limited by the type of communication and/or data transmission between the base unit 40 and a computer (not shown) or other electronic equipment. The base unit 40 may be small enough to be attached to the body of the user of the sensor 10, such as through a headband, eye patch, or biologically safe adhesive, or it may be affixed to a pair of eyeglasses or other apparel. Alternatively, the base unit 40 may be constructed as a hand held device that is not affixed to or worn by the user.

The base unit 40 may be programmed so that the base MPU 44 will send an electromagnetic pulse to the base antenna 42 at a known frequency, which it is contemplated will be a relatively low frequency. The sensor antenna 11 will be configured such that the electromagnetic pulse will cause the sensor antenna 11 to charge the power capacitor 36 with sufficient energy to power the components of the sensor 10 for one interrogation cycle. What constitutes an interrogation cycle will depend on the specific application of the sensor 10 as well as the type of pressure monitoring device employed therewith. For the embodiment shown in FIG. 4, an interrogation cycle to comprises the sensor MPU 12 supplying energy to the strain array 17, comparing the resistance between the two pairs of resistors, and transmitting that information back to the base unit 40 through the cooperative interaction between the transmitter 30 in the sensor 10 and the sensor antenna 11, and between the receiver 32 in the base unit 40 and the base antenna 42. The base unit 40 may then store the data it received from the sensor 10 in the flash memory 14 of the base unit 40. After a Is predetermined number of interrogation cycles, the information in the flash memory 14 of the base unit 40 may be transferred to a computer (not shown) through the USB interface 46. Alternatively, the information could be wirelessly transferred to another piece of equipment, such as a computer.

The sensor antenna 11 may also serve as a communication link between the sensor MPU 12 and other electronic equipment. For example, the sensor MPU 12 may be configured so that when the sensor antenna 11 and receiver 32 in the sensor 10 receive electromagnetic energy having certain characteristics (i.e., frequency, wavelength, duration, digital code, etc.), the sensor MPU 12 changes the interrogation cycle, downloads the information stored in the flash memory 14 of the sensor 10 (if so equipped), uploads a new program, performs a self-diagnostic, or performs some other function advantageous to the application for which the sensor 10 is used. Other methods may be used to program, reprogram, and/or control the sensor MPU 12 without limitation. The sensor MPU 12 may be configured to digitize the information it receives from the pressure monitoring device, in which case the sensor antenna 11 would transmit a digitized signal to the base unit 40. The base unit 40 and/or sensor 10 may require other circuitry components, such as amplifiers, resistors, capacitors, etc. for proper configuration for the specific application. Such modifications are within the purview of one of ordinary skill in the art and therefore will not be described in detail herein for purposes of clarity.

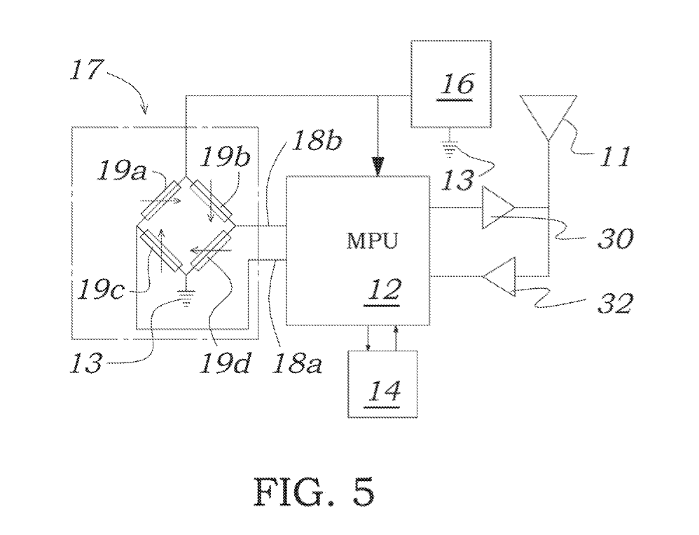

Another exemplary embodiment of the circuitry for a sensor 10 with a strain array 17 is shown in FIG. 5. The embodiment shown in FIG. 5 functions in a manner similar to that in which the embodiment shown in FIG. 4 functions. However, the embodiment shown in FIG. 5 includes a battery 16 for a power source, wherein the embodiment shown in FIG. 4 utilizes an external power source. The battery 16 may be affixed to or embedded in the body 15 of the sensor, and is electronically connected to the sensor MPU 12. Because the sensor 10 shown in FIG. 5 includes an internal power source, it is often referred to as an active sensor 10. Furthermore, because it has an to internal power source, the sensor 10 preferably includes flash memory 14 and functions as a data logger. That is, the sensor MPU 12 is programmed to perform interrogation cycles at predetermined time intervals. Each interrogation cycle produces a data point from the strain array 17, which is proportional to IOP at the time the interrogation cycle was performed. The sensor MPU 12 is programmed to store each data point in the flash memory 14. After a predetermined amount of time, the sensor antenna 11 receives electromagnetic energy with certain characteristics that causes the receiver 32 in the sensor 10 and sensor MPU 12 to transmit the data in the flash memory 14 to the transmitter 30 in the sensor 10 and the sensor antenna II, which data may be received by a base unit 40 or other electrical equipment, such as a computer (not shown).

As previously described, the strain array 17 that is used as a pressure monitoring device in the embodiments of the sensor 10 shown in FIGS. 4 and 5 is comprised of four resistors 19a, 19b, 19c, 19d that have a resistance that varies based on deformation of the resistor 19a, 19b, 19c, I 9d. However, other strain arrays 17 may be used with the sensor 10 without departing from the spirit or scope of the present disclosure. Accordingly, any strain array 17 known to those skilled in the art that is suitable for the particular application of the sensor 10 may be used without limitation. Furthermore, any pressure monitoring device that changes an electrical property in response to IOP may be used without limitation. For example, in an embodiment not pictured herein, the pressure monitoring device is comprised of a plurality of pressure sensitive resistors that are electronically connected to the sensor MPU 12. In this embodiment, the voltage drop across the plurality of pressure sensitive resistors may be correlated to IOP. Other variations will occur to those skilled in the art without departing from the scope of the sensor 10 as described and claimed herein.

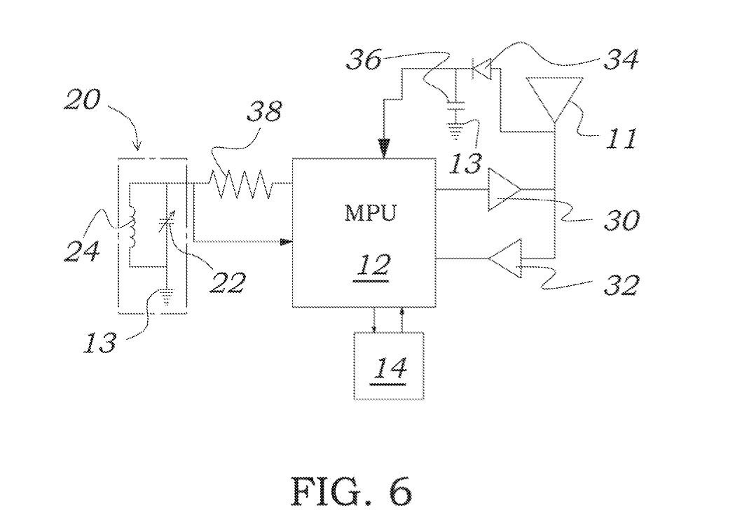

Another embodiment of a sensor 10 is shown in FIG. 6. In FIG. 6 the pressure monitoring device is comprised of a resonant circuit 20. The resonant circuit 20 is comprised of an inductor 24, variable capacitor 22 (which also may be a fixed capacitor in other embodiments), and a ground 13. An MPU resistor 38 is electronically connected to both the sensor MPU 12 and the resonant circuit 20. As with the embodiment of a sensor 10 using a strain array 17 as a pressure monitoring device, embodiments of the sensor 10 using a resonant circuit 20 as a pressure monitoring device may include a internal power source such as a battery 16, or the sensor 10 may be powered through an external signal through the sensor antenna 11.

In the embodiment in FIG. 6, the resonant circuit 20 is configured so that the resonant frequency varies proportionally to IOP. There are many ways in which this may be accomplished, and therefore any configuration of a resonant circuit 20 known to those skilled in the art that may be made so that its resonant -frequency varies in proportion to IOP may be used without limitation. For example, if the variable capacitor 22 is comprised of two dielectric plates, wherein one plate abuts the sclera 4, as the capacitance of the variable capacitor 22 increases, the resonant frequency increases, which frequency may then be correlated to IOP.

The interrogation cycle for the embodiment of the sensor 10 shown in FIG. 6 wherein the sensor 10 does not include an internal power source (such as a battery 16) varies from the interrogation cycle for the embodiment shown in FIG. 4. When a resonant circuit 20 is used as a pressure monitoring device, the resonant frequency is the quantity that may be correlated to IOP. Accordingly, the sensor MPU 12 is programmed to subject the resonant circuit 20 to energy of varying frequencies using the sensor MPU 12 in a continuous, preferably sinusoidal manner an monitor the output from the resonant circuit 20. The sensor MPU 12 will detect the resonant frequency of the resonant circuit 20 and either record the data point in flash memory 14 (if equipped) or transmit the data point to a base unit 40 in a manner similar to that described above. As is apparent to those skilled in the art, a resonant circuit 20 may be used as the pressure monitoring device whether the sensor 10 includes an internal or external power source. A variable capacitance resonant circuit 20 such as the one described above may also include a variable resistance element, such as a pressure sensitive resistor to increase the accuracy and/or precision of the resonant circuit 20.

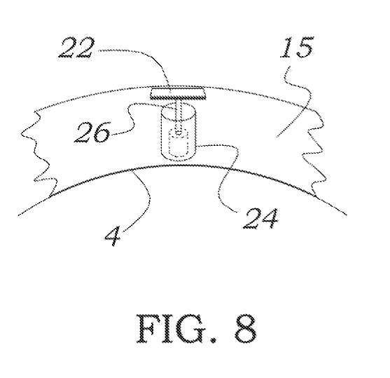

Another embodiment of a sensor 10 employing a resonant circuit 20 as the pressure monitoring device is shown in FIG. 8. In that embodiment, the resonant circuit includes a variable capacitor 22 and a variable inductor 24. The variable inductor 24 consists of an inductive coil having a ferrous member 26 positioned therein. As the position of the ferrous member 26 changes with respect to the inductive coil, the inductance of the inductor 24 changes. The ferrous member 26 may be mechanically connected to one of the plates of the variable capacitor 22 such that the change in capacitance and the change in inductance of the resonant circuit 20 are coupled. As implied, one type of variable capacitor 22 that may be used with the embodiment shown in FIG. 8 is comprised of two plates separated by a dielectric, which type of variable capacitor is well known to those skilled in the art and therefore will not be described in detail herein. Accordingly, a change in IOP would produce both a change in capacitance and a change in inductance, which together would have a greater effect on the resonant frequency than a change in either variable alone would have. This is true because an increase in capacitance yields a decrease in resonant frequency, and an increase in inductance yields a decrease in resonant frequency. Other combinations and/or configurations of electrical components known to those skilled in the art may be used to correlate a change in resonant frequency with a value for IOP without departing from the spirit and scope of the present disclosure. Accordingly, all embodiments pictured and described herein are for exemplary purposes only and are in no way meant to be limiting.

Other types of pressure monitoring devices may be used with the sensor 10 other than a strain array 17 and a resonant circuit_{.} 20 as pictured and described herein. For example, piezoelectric pressure transducers may be used, as well as thermistors, piezo-resistive transducers, silicon strain gauges, semiconductor devices and the like may be used as the pressure monitoring device. Accordingly, any electrical component that responds in a detectable manner in proportion to IOP may be used with any embodiment of the sensor 10 without limitation.

Any of the embodiments of the sensor 10 as disclosed and described herein that include a sensor MPU 12 may also be used without a sensor MPU 12. In such an embodiment, the sensor 10 would not be a smart sensor 10. Instead, a sensor 10 without a sensor MPU 12 would only measure IOP when directed to do so by an external source. For example, a sensor 10 with a resonant circuit 20 for a pressure monitoring device and no sensor MPU 12 may be interrogated with a grid dip meter (not shown) to find the resonant frequency. As described above, the resonant frequency may then be correlated to IOP, the manner of which is dependent upon the configuration of the resonant circuit 20 (i.e., variable capacitance, variable resistance, variable inductance, or combinations thereof). In embodiments of the sensor 10 wherein a sensor MPU 12 is not used, it is contemplated that the sensor 10 should be placed within the periphery of a substantially circular interrogation device (e.g., grid dip meter, electromagnetic field generator, etc.) so that the effect the distance between the sensor 10 and the interrogation device has on the resonant frequency is nullified. In an embodiment not shown, the interrogation device is disposed in the frame of a pair of eyeglasses wherein an inductive coil is positioned around the periphery of each lens.

In another embodiment not pictured herein, the sensor 10 may be used to deliver a predetermined amount of medication upon a given value of IOP. In such an embodiment the sensor 10 would further comprise a delivery switch that would function to cause a predetermined amount of medication to be delivered to a specific location from a medication storage area. It is contemplated that both the delivery switch and the medication storage area may be affixed to or embedded in the body 15 of the sensor 10. However, the delivery switch and the medication storage area may be external to the sensor 10 and in remote communication therewith. If located within the sensor 10, the medication storage area may be configured as a bladder. The delivery switch may be configured as a valve between the medication storage area and the eye 2. In operation, when the pressuring measuring device measures an elevated IOP, the sensor MPU 12 may be programmed to direct the delivery switch to open, thereby releasing a predetermined amount of medication to the eye.

If the medication storage area and delivery switch are external to the sensor 10, they may be in communication with an intra venous (IV) system. For example, the medication storage area may be configured as an IV bag plumbed to a typical IV system and the delivery switch may be configured as a valve affixed to the IV bag. The valve would be in communication, most likely wirelessly, with the sensor 10 so that the valve would open upon certain instructions transmitted from the sensor 10. Alternatively, the medication storage area may be configured is a punctual insert that is in direct communication with the sensor 10 such that the punctual insert releases a predetermined amount of medication based on direction from the sensor 10. Any of the embodiments described herein for medication delivery would allow for instantaneous medication treatment of elevated IOP.

An infinite number of configurations and/or arrangements of the components of the sensor 10 including the sensor MPU 12, sensor antenna 11, various grounds 13, flash memory 14 (if so equipped), power source (if so equipped), pressure monitoring device, transmitter 30, receiver 32, power capacitor 36 (if so equipped), MPU resistor 38 (if so equipped), and any other circuitry components as well as the electronic connections therebetween exist. Modifications of these design factors, as well as the specific configuration of the sensor MPU 12 and internal circuitry thereof, in no way limit the scope of the present disclosure. Similarly, an infinite number of configurations and/or arrangements of the components of the base 40 including the base MPU 44, base antenna 42, USB interface 46, transmitter 30, receiver 32, and any other circuitry components as well as the to electronic connections therebetween exist. Modifications of these design factors, as well as the specific configuration of the base MPU 44 and internal circuitry thereof, in no way limit the scope of the present disclosure.

The materials used to construct the sensor 10 and various electrical components thereof may be any suitable material known to those skilled in the art that is suitable for the particular. application of the sensor 10. For example, the sensor MPU 12 may be constructed of a fiberglass substrate with copper or gold traces. Accordingly, the materials of construction for the sensor 10 or the various components thereof in no way limit the scope of the present disclosure.

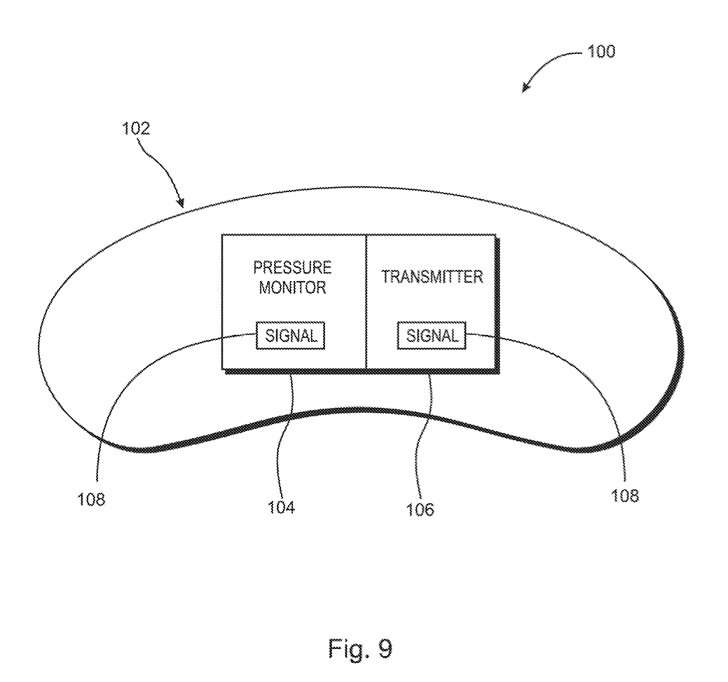

FIG. 9 is a perspective top view of sensor 100.

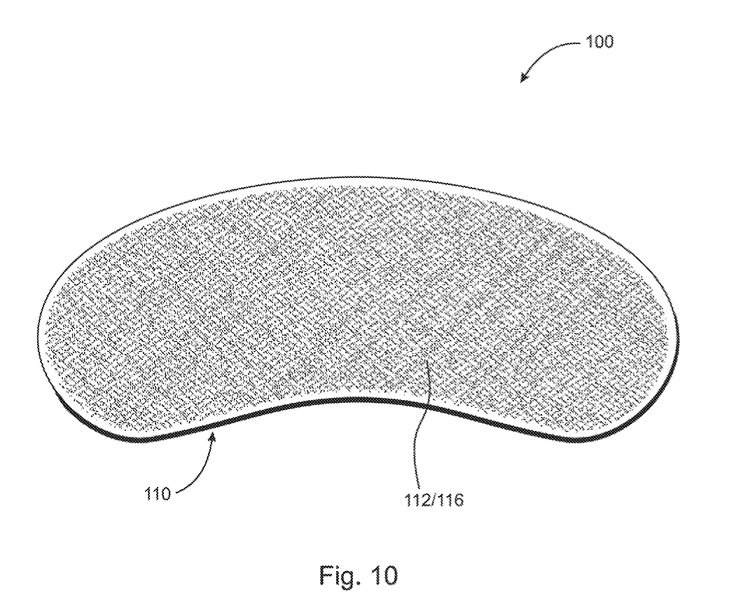

FIG. 10 is a perspective bottom view of sensor 100 shown in FIG. 9.

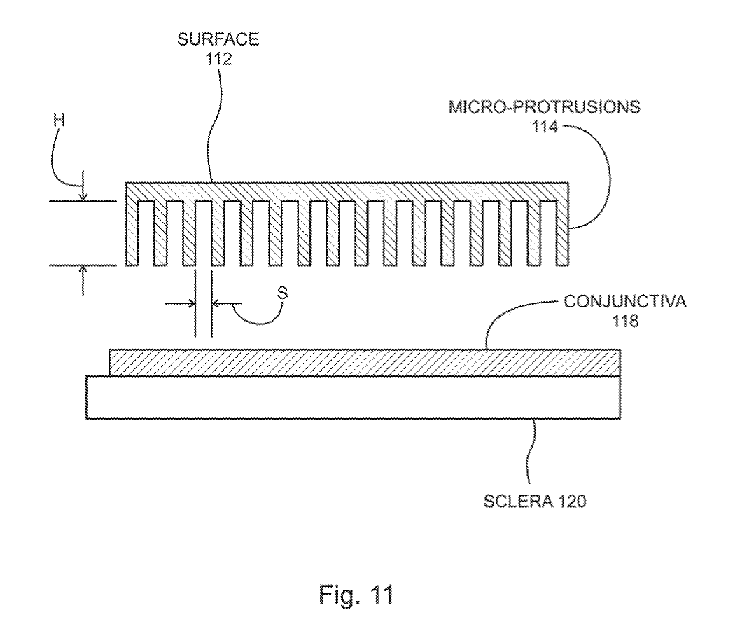

FIG. 11 is a schematic representation of micro-protrusions.

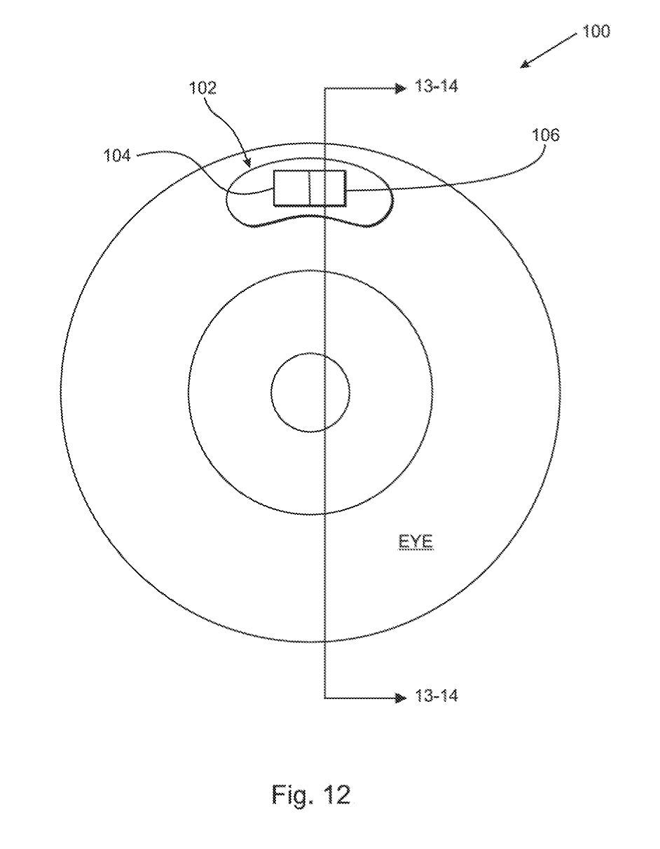

FIG. 12 is a perspective view of sensor 100 shown in FIG. 9 adhered to a human eye.

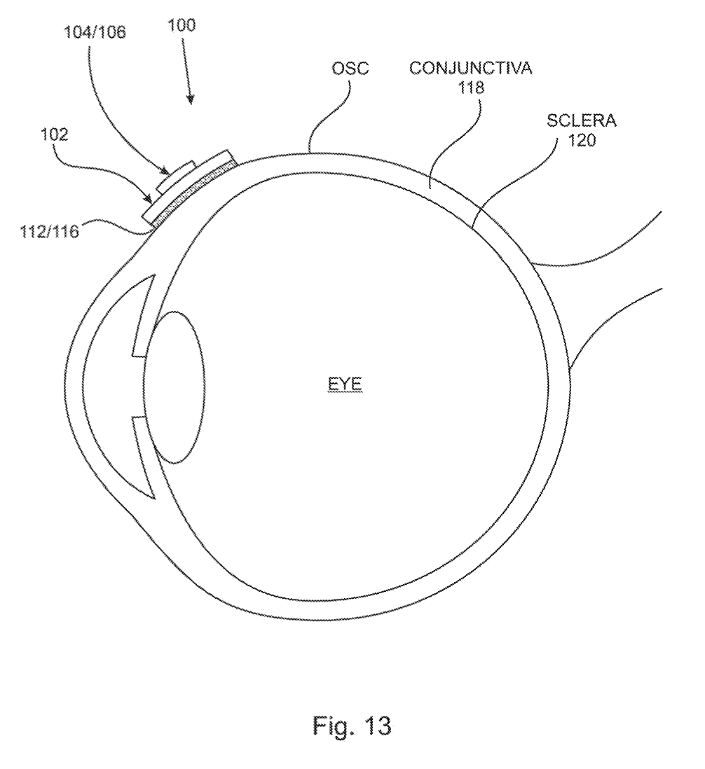

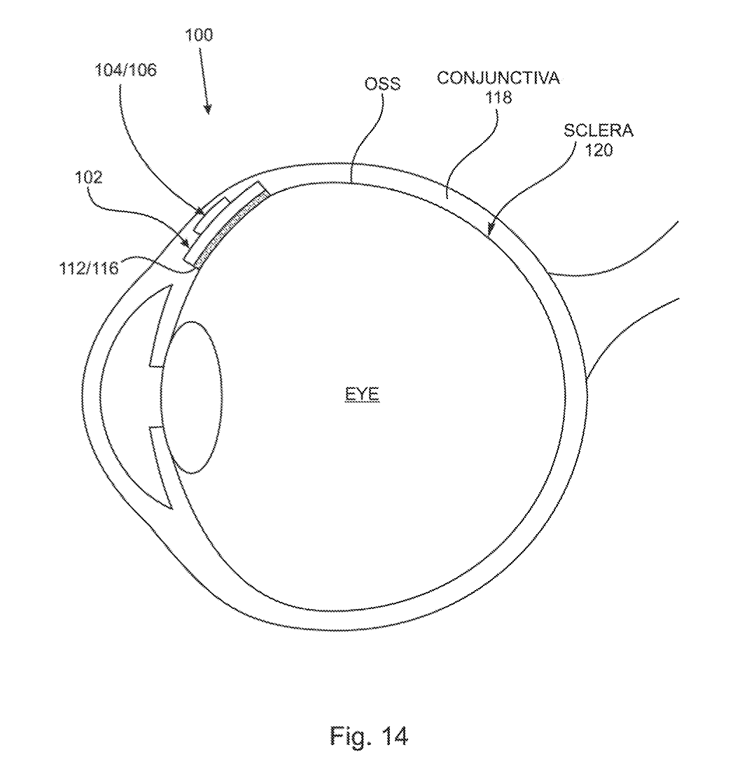

FIG. 13 is a cross-sectional view generally along line 13-14/13-14 in FIG. 12.

FIG. 14 is a cross-sectional view generally along line 13-14/13-14 in FIG. 12. The following should be viewed in light of FIGS. 9 through 14. Sensor 100 includes body 102, pressure monitoring device 104, and transmitter 106. The pressure monitoring device is configured to measure intraocular pressure of an eye and generate signal 108 representing the measured pressure. The transmitter is configured to transmit the signal. In an example embodiment, the body includes bottom surface 110 with adhesion surface 112 with a plurality of micro-protrusions 114. In an example embodiment, the micro-protrusions are made of patterned silicone. In an example embodiment, the sensor includes bottom surface 110 with adhesive 116 adhered to bottom surface 110. In an example embodiment, the bottom surface, for example, surface 112 and the plurality of micro-protrusions or adhesive 116, is arranged to secure the sensor, for example, body 102, to outer surface OSC of conjunctiva 118 for the eye without penetrating OCS or sclera 120 for the eye, for example as shown in FIG. 13. In this example embodiment, no portion of sensor 100 penetrates OSC and thus, no portion of sensor 100 penetrates the conjunctiva or the sclera. In an example embodiment, the bottom surface, for example, surface 112 and the plurality of micro-protrusions or adhesive 116, is arranged to secure the sensor, for example, body 102, to outer surface OSS of the sclera without penetrating the sclera, for example as shown in FIG. 14. In this example embodiment, no portion of sensor 100 penetrates OSS and thus, no portion of sensor 100 penetrates the sclera. Sensor 100 does not contact the cornea of the eye.

Body 102 includes top surface 122 and in an example embodiment, the pressure monitoring device and the transmitter are secured to surface 122 by any means known in the art. In an example embodiment, top surface 122 including device 104 and the transmitter is smooth. In an example embodiment, the bottom surface includes both the adhesion surface and the adhesive and the plurality of micro-protrusions and the adhesive are arranged to secure the sensor to outer surface OSC without penetrating the conjunctiva or the sclera, or to secure the sensor to outer surface OSS without penetrating the sclera.

An adhesive attraction between the adhesion surface and the eye, for example, OSC. or OSS, is dependent upon a height H of each micro-protrusion in the plurality of micro-protrusions and a spacing S of micro-protrusions in the plurality of micro-protrusions. The adhesion attraction secures the body to OSC or OSS when sensor 100 includes surface 112. In an example embodiment, H is from 3 to 40 microns and spacing S is from 3 to 40 microns. It should be understood that other respective values of H and S are possible.

Adhesive 116 can be any medical grade, or biologically safe adhesive known in the art. In an example embodiment, the adhesive is reversible, that is, the sensor can be removed from the conjunctiva or the sclera without damaging the conjunctiva or sclera. Adhesive 116 can include, but is not limited to silicones, hydrogels, polymer hydrogels, and poly-methyl methacrylates. Body 102 can be made of or can be encased in any biologically safe material known in the art, including, but not limited to materials used for constructing contact lenses, silicones, hydrogels, polymer hydrogels, and poly-methyl methacrylates.

The pressure monitoring device and the transmitter can be any pressure monitoring device and transmitter, respectively, known in the art. Any type of material known in the art, including, but not limited to silicone, can be used for surface 112 and micro-protrusions 114. The size, shape, and configuration of micro-protrusions can be any applicable size, shape, and configuration known in the art.

Advantageously, neither adhesive 116 nor micro-protrusions 114 break or penetrate respective surface tissue for OSC or OSS. Thus, sensor 100 is non-invasive and sensor 100 does not to damage the tissue to which it is secured, for example, the conjunctiva or sclera.

The present disclosure also includes a method for measuring intraocular pressure using sensor 100. One step secures, with the bottom surface, the sensor to: the outer surface of the conjunctiva of an eye without penetrating the conjunctiva or a sclera for the eye; or the outer surface of a sclera for an eye without penetrating the sclera. Another step measures, using the pressure monitoring device, intraocular pressure in the eye. A further step generates a signal representing the measured pressure. Yet another step transmits, using the transmitter, the signal.

In an example embodiment, the adhesive is separately formed from the body as an adhesion substrate and is separately adhered to the conjunctiva or sclera. After the adhesion substrate is applied, the remainder of sensor 100 is positioned on and adhered to the substrate. In an example embodiment, sensor 100 includes MPU 12 and the discussion regarding sensor 10 and MPU 12 is applicable to sensor 100. In an example embodiment, sensor 100 includes the embodiment shown in FIG. 4 and the discussion regarding sensor 10 and FIG. 4 is applicable to sensor 100. In an example embodiment, sensor 100 includes strain array 17 and the discussion regarding FIGS. 4 and 5, sensor 10 and strain array 17 is applicable to sensor 100. In an example embodiment, sensor 100 is usable with base unit 40 and the discussion of base unit 40 and sensor 10 is applicable to base unit 40 and sensor 100. In an example embodiment, sensor 100 includes antenna 11 and the discussion regarding sensor 10 and antenna 11 is applicable to sensor 100.

In an example embodiment, sensor 100 includes resonant circuit 20 and the discussion regarding FIGS. 6 and/or 8, sensor 10 and resonant circuit 20 is applicable to sensor 100. In an example embodiment, the discussion regarding FIGS. 6, sensor 10 and an interrogation cycle is applicable to sensor 100. In an example embodiment, the pressure monitoring device can include, but is not limited to, a piezoelectric pressure transducer, a thermistor, a piezo-resistive transducer, a silicon strain gauge, and a semiconductor device. In general, any electrical component that responds in a detectable manner in proportion to IOP may be used with sensor 100.

It will be appreciated that various of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

What is claimed is:

1. A sensor comprising:
a bottom surface with:
an adhesion surface including a plurality of micro-protrusions; or,
an adhesive adhered to the bottom surface;
a pressure monitoring device configured to measure intraocular pressure of an eye and to generate a signal representing the measured pressure; and
a transmitter configured to transmit the signal, wherein the bottom surface is arranged to secure the sensor to:
an outer surface of a conjunctiva for the eye without penetrating the conjunctiva; or,
an outer surface of a sclera for the eye without penetrating the sclera.

2. The sensor of Claim I wherein the bottom surface includes only the adhesion surface and not the adhesive.

3. The sensor of claim 2 wherein the bottom surface is arranged to secure the sensor to the outer surface of the conjunctiva and not to the outer surface of the sclera.

4. The sensor of claim 2 wherein the bottom surface is arranged to secure the sensor to the outer surface of the sclera and not to the outer surface of the conjunctiva.

5. The sensor of Claim I wherein the bottom surface includes only the adhesive and not the adhesion surface.

6. The sensor of claim 5 wherein the bottom surface is arranged to secure the sensor to the outer surface of the conjunctiva and not to the outer surface of the sclera.

7. The sensor of claim 5 wherein the bottom surface is arranged to secure the sensor to the outer surface of the sclera and not to the outer surface of the conjunctiva.

8. The sensor of claim 1 wherein the bottom surface includes both the adhesion surface and the adhesive.

9. The sensor of claim 8 wherein the bottom surface is arranged to secure the sensor to the outer surface of the conjunctiva and not to the outer surface of the sclera.

10. The sensor of claim 8 wherein the bottom surface is arranged to secure the sensor to the outer surface of the sclera and not to the outer surface of the conjunctiva.

11. The sensor of claim 1 wherein:
an adhesive attraction between the sensor and the eye is dependent upon a height of each micro-protrusion in the plurality of micro-protrusions and a spacing of micro-protrusions in the plurality of micro-protrusions; and,
the bottom surface is arranged to secure the sensor to the outer surface of the conjunctiva or the outer surface of the sclera using the adhesive attraction.

12. The sensor of claim 1 wherein the bottom surface is arranged to secure the sensor to the outer surface of the conjunctiva and not to the outer surface of the sclera.

13. The sensor of Claim I wherein the bottom surface is arranged to secure the sensor to the outer surface of the sclera and not to the outer surface of the conjunctiva.

14. A sensor comprising:
an adhesion surface with a plurality of micro-protrusions;
a pressure monitoring device configured to measure intraocular pressure of an eye and generate a signal representing the measured pressure; and
a transmitter configured to transmit the signal, wherein the adhesion surface is arranged to secure the sensor to an outer surface of a conjunctiva for the eye without penetrating the conjunctiva.

15. A method for measuring intraocular pressure using a sensor including a bottom surface with an adhesion surface with a plurality of micro-protrusions or an adhesive adhered to the bottom surface, a pressure monitoring device, and a transmitter, the method comprising:
securing, with the bottom surface, the sensor to:
an outer surface of a conjunctiva for an eye without penetrating the conjunctiva; or,
an outer surface of a sclera for an eye without penetrating the sclera;
measuring, using the pressure monitoring device, intraocular pressure in the eye;
generating a signal representing the measured pressure; and,
transmitting, using the transmitter, the signal.

16. The method of claim 15 wherein securing, with the bottom surface, the sensor to the outer surface of the conjunctiva or the outer surface of the sclera includes securing the sensor only to the outer surface of the conjunctiva.

17. The method of claim 15 wherein securing, with the bottom surface, the sensor to the outer surface of the conjunctiva or the outer surface of the sclera includes securing the sensor-only to the outer surface of the sclera.

18. The method of claim 15 wherein securing, with the bottom surface, the sensor to the outer surface of the conjunctiva or the outer surface of the sclera includes securing the sensor to the outer surface of the conjunctiva or the outer surface of the sclera only with the adhesion surface.

19. The method of claim 15 wherein securing, with the bottom surface, the sensor to the outer surface of the conjunctiva or the outer surface of the sclera includes securing the sensor to the outer surface of the conjunctiva or the outer surface of the sclera only with the adhesive.

20. The method of claim 15 further comprising generating an attraction force according to a height of each micro-protrusion in the plurality of micro-protrusions and a spacing of micro-protrusions in the plurality of micro-protrusions, wherein securing, with the bottom surface, the sensor to the outer surface of the conjunctiva or the outer surface of the sclera includes securing the sensor with the attraction force.
